(19) United States
(12) Patent Application Publication
(10) Pub. No.: US 2003/0211459 A1
(43) Pub. Date: Nov. 13, 2003

(54) Drug development by rapid neuroimaging of neural cells

(75) Inventors: Hans Breiter, Lincoln, MA (US); David Borsook, Concord, MA (US)

Correspondence Address: DALY, CROWLEY & MOFFORD, LLP, SUITE 101, 275 TURNPIKE STREET, CANTON, MA, 02021-2310, US

(73) Assignee: The General Hospital Corporation, Boston, MA

(21) Appl. No.: 10/279,413

(22) Filed: Oct. 24, 2002

Related U.S. Application Data
(60) Provisional application No. 60/351,205, filed on Oct. 25, 2001.

(51) Int. Cl. C12Q 1/00; G06F 19/00; G01N 33/48; G01N 33/50; G06K 9/00; C12N 5/08

(52) U.S. Cl. 435/4; 435/368; 702/19; 382/128

(57) ABSTRACT

The invention provides a method for identifying potential therapeutic agents by relating in vitro techniques for drug screening of neural cells with neural circuitry function in animals and humans. The method involves objectively measuring, in a quantifiable and reproducible manner, the effects of the agents on pain and other motivational functions.

### CLAIM OF PRIORITY

This application claims priority to U.S. provisional patent application Serial No. 60/351,205, filed Oct. 23, 2001.

### FIELD OF THE INVENTION

The invention relates to analytical testing, and more particularly to methods of drug development using imaging techniques.

### BACKGROUND OF THE INVENTION

For the rational and focussed development and screening of drugs and biopharmaceutical agents with neurological and psychiatric effects, one of skill in the pharmaceutical arts will need to assess how well the test agent affects the targeted functional circuitry of the brain that underlies the signs, symptoms or behavior to be altered. To date, skilled artisans have developed potential target compounds or gene products without direct evidence that these test agents affect the brain circuitry underlying the targeted signs, symptoms, or behaviors. Skilled artisans have had to assess these test agents using animal behavior models that only approximate the human conditions, and then subsequently assess the agents in humans against subjective or indirect measures of the targeted signs, symptoms, or behaviors.

The science of genomics can be used to define alterations in gene expression and function following a perturbation. For example, cutting a nerve causes the increase or decrease of expression and activity in many hundreds of genes. However, this approach is difficult, if not impossible, for the development of drugs targeting genes or gene products involved in pain or nerve damage, for two reasons. First, it is unlikely a priori that one drug or biologic will be therapeutically effective for all of the genes or gene products. Second, these peripheral changes (i.e., cutting an axon or dendrite outside of the central nervous system) do not take into account the neural circuitry of behavior, which is in the central nervous system and responds in a more general fashion to very different, but categorically related, stimuli. Clearly, a number of changes in the periphery can produce similar changes in central circuitry.

What is needed for the rational drug development of agents for treating neurologically based conditions is an objective methodology that links to brain circuitry function with the rational identification and rapid screening of the agents under investigation.

### SUMMARY OF THE INVENTION

The invention provides a method for identifying potential therapeutic agents, such as drugs and gene products (biologics), by relating in vitro techniques for drug screening of neural cells with neural circuitry function in animals and humans. The method involves objectively measuring, in a quantifiable and reproducible manner, the effects of the agents on pain and other motivational functions.

Thus, the invention provides a new tool for the rational drug development for therapeutic agent to be used in the treatment of pain, psychiatric illness and other neurologically based conditions. The invention also provides a way to correlate information from neuroimaging techniques such as functional magnetic resonance imaging (fMRI) in animal models with data obtained from in vitro drug development data.

### BRIEF DESCRIPTION OF THE DRAWINGS

FIG. 1 is a flow chart organizing techniques for target compound screening and development using the regional tissue neuroimaging technique (RTNT) and other established drug screening and development methods. FIG. 1A is a flow chart of the use of RTNT with tissue slices (or neural cell culture) or in vivo recording in conjunction, with screening of target compounds via gene expression effects along with screening of in vivo effects using neuroimaging. The candidate evaluation process evaluates compound effects at multiple scales of brain function (i.e., genetic, neural, neural group, and distributed neural group). FIG. 1B is a flow chart of the use of RTNT in conjunction with other screening methods over the course of drug development from discovery phases, to preclinical and clinical phases.

FIG. 2 is a flow chart of the regional tissue neuroimaging technique (RTNT) of the invention.

### DETAILED DESCRIPTION OF THE INVENTION

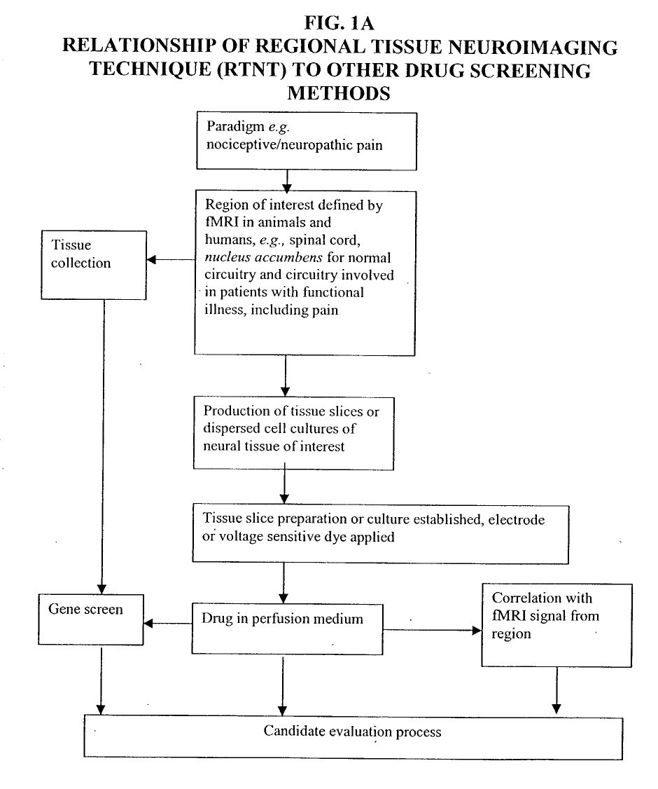

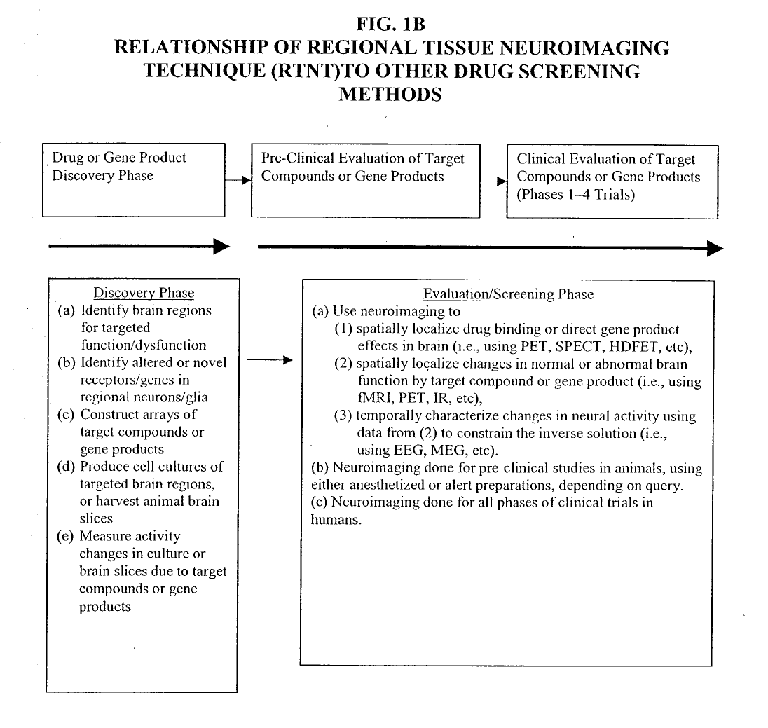

Purpose of the invention. Prior to this invention, there have been no objective markers for pain or functional illness to use for the rational development of targeted drugs or gene products (biologics) to address these neurologically-based conditions. To rationally develop such agents for these neurologically-based conditions, one of skill in the pharmaceutical arts should be able to have an objective ability to assess how well a compound, at each level of drug development (for example, see steps in FIG. 1A and FIG. 1B) affects the targeted functional circuitry of the brain that is involved with a particular condition. The skilled artisan should be able to obtain similar measurements from neural cells using techniques that are amendable to rapid screening of many compounds and using techniques are indicative of functional circuitry. Brain circuitry directly underlies behavior and the signs of illness reported by human patients or observed by physicians.

The recent development of the science of genomics has resulted in the discovery of many potential targets for therapy of pain and dysfunctions of motivational function (including psychiatric illnesses). However, the typical genomics bottom-up approach (i.e., from gene to behavior) has significant difficulty with determining a priori whether or not a gene or potential drug will have the desired effect on pain or psychiatric illness. Without an intermediary link via measurements of brain circuitry, the skilled artisan cannot efficiently screen enough potential drug candidate compounds or gene products (biologics) to get a sufficient set that will affect the targeted sign, symptom, or behavior.

By contrast, functional imaging of circuitry permits a top-down approach to linking behavior and molecular/receptor/gene function. Neuroimaging of the circuitry of reward in humans (Breiter et al., Neuron 30(2):619-639 (2001); Breiter et al., Neuron 19(3):591-611 (1997)) has determined that there exists a generalized circuitry for reward, at the spatial scale of distributed neural groups, that responds to multiple categories of stimuli, including very different drugs of abuse (i.e., cocaine vs. morphine), money, and social stimuli such as beautiful faces. This system appears to constitute a generalized one for assessing the relevant features from goal-object information for the organization of behavior. This general system for the assessment of reward information also mediates the processing of aversive information. Recent work also shows that the “classic” circuitry of reward also activates in a unique way to aversive stimuli (Becerra et al., Neuron 32:927-946 (2001)), and that the “classic” circuitry of pain is also present during stimulation with rewarding goal-objects.

Neural circuitry of motivation. The assessments of rewarding and aversive information are at the core of motivation function. To produce behavior, motivational states necessitate at least three fundamental operations, including (1) selection of objectives that optimize fitness over time, (2) compilation of information about potential goal-objects to meet these objectives or to avoid to meet these objectives, and (3) determination of physical plans for securing or avoiding selected goal-objects (i.e., rewards and aversive events). The second of these general operations is an informational backbone for motivation (iBM) and involves the extraction of information features from perceptual representations such as the rate, latency, incidence, intensity, amount, category, and proximity of the reinforcing or aversive stimuli. The general system for the assessment of rewarding and aversive information constitutes this “iBM”.

All behavior is organized on the basis of the functional output of this “iBM”. The signs, symptoms, and behavior that are the targets of medication treatment in neurology and psychiatry depend on the function of this iBM. Neuroimaging of this iBM provides a fundamental, but not the only, target for assessment of changes in the signs, symptoms, and behavior targeted by medication. Neuroimaging of this iBM and other regions (e.g., imaging somatosensory cortex in the objective diagnosis of pain; this region is not properly part the iBM) could provide an objective test for activity as a component of drug assessment (see, FIG. 1B).

Neuroimaging methods. A powerful means of assessing brain circuitry in vivo in animals or humans is “neural imaging” or “neuroimaging” that assesses measures related to activity in distributed neural groups, via methods such as functional magnetic resonance imaging (fMRI; see, U.S. Pat. Nos. 6,275,723, 6,298,258 and 6,306,077, incorporated herein by reference), PET, SPECT, HDFET, electroencephalography (EEG), MEG, optical imaging, etc. These neuroimaging tools appear to bring increased power to the assessment and evaluation of drugs and gene products that have been developed (see, FIG. 1).

fMRI can use the blood oxygen level dependent (BOLD) effect to determine activation within brain regions of humans and animals during specific experimental conditions. The BOLD effect specifically, in parallel with other functional neuroimaging methods such as PET, SPECT, HDFET, EEG, MEG, and optical imaging, provide objective determination of functional circuits in the brain. The BOLD signal measured by fMRI is strongly related to alterations in local field potentials (see, Logothetis NK et al., Nature 412, 150-157, (2001); Raichle M E, Nature 412, 128-130 (2001)), as is, to some degree, the signal related to cerebral blood flow picked up by some applications of positron emission tomography (PET), Single-Photon Computed Tomography (SPECT); High-Definition Focusing Emission Tomography (HDFET), and optical imaging. Currently, however, the measurement of these signals in vivo (i.e., in animals or humans) is relatively time-intensive for the beginning development of drugs or gene products (biologics).

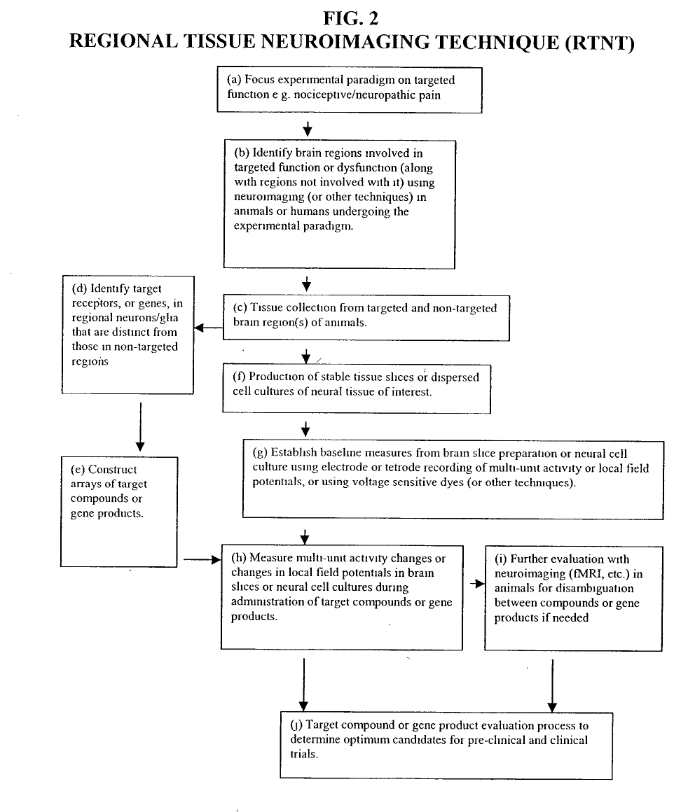

Recent work by Logothetis N K et al., Nature 412, 150-157, (2001) points to the fMRI signal (i.e., BOLD signal) being predominantly due to local field potentials of neural populations. This dendritic and cell soma based information processing leads to, but is distinct from, the process of action potential generation, that is a necessary aspect of information communication between cells. It is noteworthy that the same local field potentials help determine the cerebral blood flow signal picked up by some forms of PET, SPECT, HDFET, and optical imaging. Local field potentials can also be measured in brain slice preparations or distributed neural cultures (FIG. 2), though they will not necessarily be modulated in the same way by endogenous neurotransmitters such as dopamine since long projection tracts from the brainstem and other sources will be missing. The fact that local field potentials, among the many types of signals related to neuron information processing that can be measured in cultured cells, or in brain slices, can be also observed by extension in functional neuroimaging of animal and human brains, means that a similar type of signal can be measured from the beginning of drug (and gene product) development to the completion of drug (and gene product) assessment. One may thus use similar measures (determined by distinct technologies) for aspects of drug discovery and aspects of drug assessment (FIG. 1B).

Drug discovery method of the invention. In contrast to genomics, the top-down method of the invention defines the effects of a drug or gene product (biologic) on the targeted condition in terms of its alteration of brain circuitry function, at a considerable savings of time and costs. The top-down approach (focussing on moving from behavior to circuitry and then to gene/molecule/receptor) addresses this inefficiency in drug development (including failures uncovered late in drug development). The top-down approach uses time-efficient, objective neuroimaging methods to discover new potential candidate drugs (i.e., target compounds) or to evaluate their efficacy for clinical trials.

The method of the invention is also advantageous, because the method can provide the objective radiological definition of a functional, neurologically based condition (the illness, signs, symptoms, and behavior targeted), even when such a definition does not pre-exist based upon alternative criteria. Thus, the method of the invention can result in a time and cost savings in the diagnosis and treatment of pain and psychiatric conditions, particularly during preclinical drug development and when assessing clinical efficacy against market standards.

The method of the invention involves identifying potential target compounds or gene products (biologics) for brain related clinical problems by obtaining a collection of neural cells (for example, neurons and glia in brain slices or dissections) from targeted brain regions and matched non-targeted regions for assessment of receptors, transmitters, genes, and other biological molecules that differentiate these neural cells and could relate to their differential brain function. Such assessments may incorporate any number of proteomic approaches such as those using MALDI Time-of-Flight Mass Spectroscopy for identification of molecular structures of biological material unique to those regions.

For drug discovery (see, FIG. 1 and FIG. 2), the top-down method of the invention begins with the identification of target brain regions involved in the function to be treated. The targeted brain regions are pre-identified by one of skill in the art based on knowledge of brain circuitry of other neurobiology. For instance, one of skill in fMRI could scan an animal brain during an experiment involving painful and non-painful stimuli to localize the brain regions and their constituent cellular components that respond to the painful input more than non-painful sensory input. Or, one could scan an animal during a model of allodynia or some other experimental paradigm relating to chronic pain. Alternatively, for guidance, one of skill in the art could refer to the published scientific literature, or scan humans with particular conditions and structurally identify homologous regions in animals to those identified from the human scanning to be important for the neurological, psychiatric, or pain issue being studied. In the case of pain, the skilled artisan could identify the somatosensory cortex, where pain signals are first received and represented in the brain. Morphine has one of its effects based on suppressing somatosensory cortex and all other cortex, so that patients taking morphine have altered attention, memory and perceptual awareness of sensory input.

Cells from these identified brain regions, contrasted with cells from regions not involved with the function under consideration, can be used to identify the differential receptors, transmitters, genes, and other biological molecules involved with the targeted function. These differential receptors, transmitters, genes, and other biological molecules can be used to develop arrays of compounds or gene products with agonist, antagonist, or other effects.

In one embodiment, a set of cells (animal cells, such as mammalian or other vertebrate cells, such as human cells) are first dissected from these regions and submitted for molecular assessment. Molecules that could interact with the genes or proteins (such as receptors) on these cells (“target compounds” or “test compounds”) are obtained or developed. The neural cells are then either grown in cell culture or harvested as brain slices to produce a system on which target compounds are tested. In a preferred embodiment, many target compounds (possibly on the order of 100,000 target compounds) are tested in an array or in high throughput screening. A signal (such as a signal commonly measured in drug development assays, such as light emitted from a reporter gene product, such as luciferase) is measured from these cells to determine if these compounds are having effects is the same as that later measured with neuroimaging (such as fMRI, see, below) in animals or humans. The target compounds found to appropriately alter neural cell function in such an assay are then tested with animal fMRI to determine if they affect any of the circuitry involved with pain or other function tested, or reduce the pain signal in these regions during experimentally induced pain.

In one embodiment, such an approach can be continued from pre-clinical screenings through clinical evaluations using fMRI in humans (see, FIG. 1B).

In another embodiment, neuroimaging of these potential drugs or gene products (biologics) can be performed using neural slices or dispersed cell cultures from the targeted brain regions (i.e., circuitry of interest identified via neuroimaging, for example). In this embodiment, brain cells are collected from the targeted circuitry of animals, either as slices or plated onto culture dishes. These brain slices or dispersed cell cultures are then interrogated (i.e., measured in a drug development assay for screening the effects of drugs on these regions) using rapid detection methods (focussed on measuring local field potentials or other related phenomena). Screening techniques are based on methods that include but are not limited to: (a) fast multi-site optical imaging/recording of slices or neural cell cultures; (b) fixed microelectrode arrays, including tetrodes; and (c) voltage sensitive dyes. For example, a voltage sensitive dye can be Di8-ANEPPS (Molecular Probes) optionally used in conjunction with Cascade Blue (Molecular Probes) to reveal cell morphology. See also, Antic S et al., Biological Bulletin 183: 350-351 (1992).

An advantage of this rapid screening methodology is that it is an in vitro method that provides a measurement that is similar to that used with fMRI in animals and humans. Specifically, when local field potential measures are made of brain slice preparations containing a targeted brain region, this measurement has a direct relationship to the in vivo fMRI measurement made using BOLD signals that also relate to local field potentials of active neural cells. In this fashion, drug discovery methods at the cellular level can be nearly seamlessly integrated with measurements related to functional neuroimaging with fMRI or other modalities.

Correlation of fMRI data obtained in vivo and in vitro; method of the invention. Neuroimaging of interactions between drugs and in vitro cells may offer further advances on looking at brain circuitry function to complement or replace the use of fMRI or other current functional imaging modalities. In this method, fMRI neuroimaging data obtained from measuring the interaction of the obtained set of in vitro neural cells contacted with a test agent is correlated with the fMRI neuroimaging data obtained in vivo (either from published data or independently). A correlation by one of skill in the art between the fMRI neuroimaging data obtained in vitro after contact with the test agent and the fMRI neuroimaging data obtained in vivo identifies the test agent as being an agent for the treatment of a brain-based condition.

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. A method for identifying an agent that can be used to treat a neurologically-based condition, comprising the steps of:
(a) obtaining a set of in vitro neural cells;
(b) obtaining data from contacting the set of in vitro neural cells with an agent suspected of being an agent that can be used to treat the neurologically-based condition;
(c) obtaining neuroimaging data from neural cells corresponding to the set of in vitro neural cells, wherein the corresponding cells are contacted with the agent suspected of being an agent that can be used to treat the neurologically-based condition; and
(d) correlating the data obtained from contacting the set of in vitro neural cells with the agent with the neuroimaging data, wherein a correlation between the data obtained from contacting the set of in vitro neural cells with the agent and the neuroimaging data identifies the agent as an agent that can be used to treat the neurologically-based condition.

2. The method of claim 1, further comprising the step of:
(e) correlating the data obtained from contacting the set of in vitro neural cells with the agent with data regarding the activity of in vivo neural cells.

3. The method of claim 1, further comprising the step of:
(e) selecting the agent for use in clinical trials.

4. The method of claim 1, wherein the neurologically-based condition is a condition that involves the neural circuitry of motivational function.

5. The method of claim 1, wherein the neurologically-based condition is a condition selected from the group consisting of pain and psychiatric illnesses.

6. The method of claim 1, wherein the neurologically-based condition is a condition that involves the neural circuitry for reward.

7. The method of claim 1, wherein step (d) comprises correlating the neuroimaging data with a function selected from the group consisting of molecular function, protein function, receptor function and gene function.

8. The method of claim 1, wherein the in vitro neural cells are mammalian cells.

9. The method of claim 1, wherein the in vitro neural cells are human cells.

10. The method of claim 1, wherein the set of in vitro neural cells is selected from the group consisting of cells in culture, tissue slices, and dissections.

11. The method of claim 1, wherein the neuroimaging is by a method selected from the group consisting of fMRI, PET, SPECT, HDFET, EEG, MEG, and optical imaging.

12. The method of claim 1, wherein set of neural cells corresponding to the set of in vitro neural cells is an in vivo set of cells in the region of an animal corresponding to the set of neural cells.

13. The method of claim 1, wherein neural cells corresponding to the set of in vitro neural cells are in culture, in tissue slices or in dissections.

14. A method for correlating fMRI data, comprising the steps of:
(a) obtaining a set of in vitro neural cells;
(b) obtaining fMRI neuroimaging data from the interaction of the set of in vitro neural cells contacted with an agent suspected of being an agent that can be used to treat a neurologically-based condition; and
(c) correlating the fMRI neuroimaging data obtained from the interaction of set of in vitro neural cells contacted with the agent with the fMRI neuroimaging data obtained in vivo, wherein a correlation between the fMRI neuroimaging data obtained from the interaction of set of in vitro neural cells contacted with the agent and the fMRI neuroimaging data obtained in vivo identifies the agent as an agent that can be used to treat the neurologically-based condition.

15. The method of claim 14, wherein the in vivo neuroimaging data includes data regarding behavior.

16. The method of claim 14, wherein the in vivo neuroimaging data includes data regarding human behavior.

17. The method of claim 1, wherein the in vivo neuroimaging data is obtained at least in part from neural cells selected from the group consisting of cells in the spinal cord, cells of the nucleus accumbens, cells of the somatosensory cortex, cells of the informational backbone for motivation (iBM), and cells from brain circuitry involved in functional illnesses.
